(19) United States
(12) Patent Application Publication
(10) Pub. No.: US 2002/0132010 A1
(43) Pub. Date: Sep. 19, 2002

(54) Divalproex sodium dosage forms and a process for their production

(76) Inventors: Yihong Qui, Gurnee, IL (US); Todd E. Chermak, Grayslake, IL (US); Kevin R. Engh, Kenosha, WI (US); Lynn Faitsch, Libertyville, IL (US); Russell T. Slade, Lindenhurst, IL (US)

Correspondence Address: ROSS PRODUCTS DIVISION OF ABBOTT LABORATORIES, DEPARTMENT 108140-DS/1, 625 CLEVELAND AVENUE, COLUMBUS, OH, 43215-1724, US

(21) Appl. No.: 09/747,912

(22) Filed: Dec. 22, 2000

(51) Int. Cl. A61K 9/14

(52) U.S. Cl. 424/489

(57) ABSTRACT

The present invention is directed to a new process for granulating valproate compounds, such as divalproex sodium, in which the compound is melted in the absence of a binder and admixed with one or more excipeints to produce granules. The resulting dosage forms have an increased drug loading factor in the range of 75 w/w % to 90 w/w %.

The present invention is directed to new solid dosage forms of divalproex sodium, valproic acid, prodrugs of valproic acid, and analogs of valproic acid (hereinafter “valproate compounds”). Other aspects of the invention are directed to improved methods of producing these new dosage forms and to improved methods of granulating these valproate compounds.

### BACKGROUND

Compressed tablets are the most common means of administering drugs. These dosage forms have a number of advantages. They are typically less expensive to produce. They are relatively small and thus are easy for the patient to swallow. For pediatric indications, they can be formulated as chewable tablets.

The initial starting material for any tablet is the bulk drug substance (i.e. the drug). This bulk drug substance is typically present as a powder. This powder cannot be compressed directly into a tablet. It lacks sufficient binding capacity to form an agglomerate. Further, it lacks the lubricating and flowing characteristics that are also necessary in tablet manufacture.

Typically, the initial step in the manufacture of any oral dosage form is a granulation. Granulation is a process of size enlargement, whereby small particles are gathered together into larger, permanent aggregates to render them into a free-flowing state. This is accomplished by adding binders to the admixture during the granulation process. The binder helps to increase the adhesiveness of the particles and thereby facilitate the size enlargement process.

There are a variety of granulation techniques known in the art. The most common is a wet granulation. The bulk drug substance is admixed with a liquid in a vessel under agitation. A binding agent is added to the liquid to facilitate the granulation. The admixture is then dried to produce granules which may be compressed into a tablet. During the drying process, the binder serves as an adhesive to bind the particles of drug together. Examples of binding agents utilized in wet granulations include polyvinylpyrrolidone, pregelatinized starch, acacia, gelatin, sodium alginate and cellulose compounds such as HPMC or HPC. The liquid used in the granulation is often an organic solvent such as ethanol, methylene chloride, etc., or water. Wet granulation is well known in the art and is described in detail in works such as PHARMACEUTICAL DOSAGE FORMS, Tablets, Lieberman et al, Marcel Dekker, Inc. pages 148-149. Other descriptions may be found in Remington's Pharmaceutical Sciences, Fifteenth Edition pages 1583-1586 (1975).

Recently, a new granulating technique has been described in the literature. It is referred to as melt granulation. This method relies on the use of solids having a low melting point which, when mixed with a powdered formulation and heated, are transformed into a liquid and act as a binder. Upon cooling, the mixture forms a solid mass in which the powders are bound together by the binder returning to the solid state. The mass is broken and reduced to granules and compressed into tablets. Materials used as binders include polyethylene glycol 4000 (PEG), polyethylene glycol 6000, stearic acid, and various waxes. The amount of binder required in a melt granulate is greater than for conventional wet granulation, (i.e. 20 to 30%, by weight, of drug substance vs. 3-10%, by weight). A detailed description this technique is described by Lieberman et al, supra, at pages 150-151.

This technique has been applied to a number of pharmaceuticals to date. Royce et al describes the use of melt granulation to produce anti-histamine tablets containing clemastine fumarate, Drug Development and Industrial Pharmacy, 22 (9&10), 917-924 (1966). Royce et al produced a number of clemastine tablets. The variables which he studied included both the quantity and the identity of the binding agent. Binders included PEG, glycerol palmitostearate, and paraffin, in quantities of up to 30 w/w % based upon the weight of drug substance. Royce concluded that this technique was suitable for immediate release or sustained release tablets, based upon in-vitro dissolution profiles generated by his collaborators.

Similar results are reported in the literature by Schaefer et al for calcium tablets, lactose tablets, and mannitol tablets, International Journal of Pharmaceutics, Vol. 139, pages 105-159, (1996). Shaefer et al reported that suitable formulations of these entities could be produced via melt granulation. Specific results varied with the specific granulating agent, its quantity, and the temperature. Schaefer cautioned, at page 117, that at excessive temperatures, he observed the decomposition of the active ingredient.

While melt granulation is well described in the literature, it is important to emphasize that the various authors describe a common phenomena. A meltable binding agent is admixed with a drug. The admixture is heated to a temperature sufficient to melt the binding agent, while having no impact on the drug. The granulation is then carried out with this molten mass to produce particles suitable for compression into tablets.

To date, the literature does not contain any reports of a successful melt granulation being carried out in the absence of a binding agent. Nor does the literature describe a process in which the bulk drug substance is heated to its melting point to accomplish the granulation.

### BRIEF DESCRIPTION OF THE DRAWINGS

FIGS. 1-3 illustrates the comparative dimensions of a 500 mg divalproex sodium tablet produced via the prior art and both a 500 mg and 750 mg divalproex sodium tablet produced via the invention.

### SUMMARY OF THE INVENTION

In accordance with the present invention, a new process for producing solid dosage forms of valproate compounds, such as divalproex sodium, has been discovered. It has been discovered that the valproate compounds may be melt granulated without a binding agent. The valproate compound may be heated to its melting point, optionally admixed with conventional excipeints, cooled, milled and compressed directly into solid dosage forms, such as tablets.

Such dosage forms exhibit significant advantages when compared with the formulations of the prior art. Substantially greater quantities of the compound may be incorporated into these dosage forms. This produces a substantially smaller formulation, which is easier for the patient to swallow. Further, it is now possible to produce solid dosage forms containing significantly higher doses of the valproate compound than was possible with the prior art methods.

For example, with conventional wet granulation technology, it is not possible to produce a solid dosage form containing greater than 500-600 mg of divalproex sodium. Dosage forms containing higher doses of divalproex sodium are too large to be comfortably swallowed. The new melt granulation process allows for the production of dosage forms containing up to 800 mg of the divalproex sodium and more preferably about 750 mg.

These new dosage forms are small enough to be comfortably swallowed, without inducing a gag reflex.

It is important to emphasize that the new process of this invention differs from the hot melt process of the prior art in two important aspects. With conventional hot melt processes, a meltable binder is admixed with the active constituent. The admixture is heated until the binder melts, not the drug. The molten binder serves as the granulating agent and binds the individual drug particles together so that they may be compressed into tablets. First, the new process does not require the presence of a binder. Secondly, the new process requires that a substantial portion of the valproate compound actually be melted. The molten drug serves as the binding agent so that the particles of the valproate compound may be agglomerated into a compositions that may be compressed into tablets.

A further advantage of the invention is that it allows the production of solid dosage forms of valproate compounds, without the use of organic solvents. The prior art techniques for granulating valproate compounds required organic solvents, such as ethanol, as the granulating liquid. These solvents created safety concerns for workers producing the dosage forms, due to the flammability of the solvent. Further, the organic solvents create environmental drawbacks due to the difficulties associated with their disposal.

The dosage forms produced via this new process also exhibit a further advantage, in addition to reduced size and weight. Granulating with an organic solvent results in residual amounts of the organic solvent being retained in the granulate and in the final dosage form. The new melt granulation process eliminates the presence of this residual organic solvent in the granulate and resulting dosage form.

### DETAILED DESCRIPTION OF THE INVENTION

As noted above, the invention relates to new and improved dosage forms of divalproex sodium, and other valproate compounds. Further aspects of the invention also relate to methods for producing these improved dosage forms. Several valproate compounds are currently available commercially in the United States or have been described in the literature.

One such compound is valproic acid. Valproic acid may be represented by the following structure:

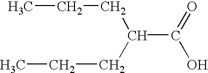

Valproic acid is available commercially from Abbott Laboratories of Abbott Park, Ill. Methods for its synthesis are described in Oberreit, Ber. 29, 1998 (1896) and Keil, Z. Physiol. chem. 282, 137 (1947). It's activity as an antiepileptic compound is described in the Physician Desk Reference, 52nd Edition, page 421, 1998.

The sodium salt of valproic acid is also known in the art as an anti-epileptic agent. It is also known as sodium valproate and is described in detail in The Merck Index, 12 Edition, page 1691, (1996). Further descriptions may be found in the Physician Desk Reference, 52nd Edition, page 417, 1998.

Divalproex sodium is effective as an antiepileptic agent, in the treatment of migraine and for bipolar disorders. Methods for its preparation may be found in U.S. Pat. Nos. 4,988,731 and 5,212,326, the contents of both which are hereby incorporated by reference.

In addition to these specific compounds, one of ordinary skill in the art would readily recognize that the carboxylic moiety of the valproic compound may be functionalized in a variety of ways. This includes forming compounds which readily metabolize in-vivo to produce valproic acid, such as valproic acid amide (valproimide), as well as other pharmaceutically acceptable amides and esters of the acid (i.e. prodrugs). This also includes forming a variety of pharmaceutically acceptable salts.

Suitable pharmaceutically acceptable basic addition salts include, but are not limited to cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Other possible compounds include pharmaceutically acceptable amides and esters. “Pharmaceutically acceptable ester” refers to those esters which retain, upon hydrolysis of the ester bond, the biological effectiveness and properties of the carboxylic acid and are not biologically or otherwise undesirable. For a description of pharmaceutically acceptable esters as prodrugs, see Bundgaard, E., ed., (1985) Design of Prodrugs, Elsevier Science Publishers, Amsterdam, which is hereby incorporated by reference. These esters are typically formed from the corresponding carboxylic acid and an alcohol. Generally, ester formation can be accomplished via conventional synthetic techniques. (See, e.g., March Advanced Organic Chemistry, 3rd Ed.,John Wiley & Sons, New York (1985) p. 1157 and references cited therein, and Mark et al. Encyclopedia of Chemical Technology,John Wiley & Sons, New York (1980), both of which are hereby incorporated by reference. The alcohol component of the ester will generally comprise (i) a C₂-C₁₂ aliphatic alcohol that can or can not contain one or more double bonds and can or can not contain branched carbons or (ii) a C₇-C₁₂ aromatic or heteroaromatic alcohols. This invention also contemplates the use of those compositions which are both esters as described herein and at the same time are the pharmaceutically acceptable salts thereof.

“Pharmaceutically acceptable amide” refers to those amides which retain, upon hydrolysis of the amide bond, the biological effectiveness and properties of the carboxylic acid and are not biologically or otherwise undesirable. For a description of pharmaceutically acceptable amides as prodrugs, see Bundgaard, H., ed., (1985) Design of Prodrugs, Elsevier Science Publishers, Amsterdam. These amides are typically formed from the corresponding carboxylic acid and an amine. Generally, amide formation can be accomplished via conventional synthetic techniques. (See, e.g., March Advanced Organic Chemistry, 3rd Ed.,John Wiley & Sons, New York (1985) p. 1152 and Mark et al. Encyclopedia of Chemical Technology,John Wiley & Sons, New York (1980)., both of which are hereby incorporated by reference. This invention also contemplates the use of those compositions which are both amides as described herein and at the same time are the pharmaceutically acceptable salts thereof.

Analogs of valproic acid have also been described in the literature. For example, biological activity has been retained in compounds in which one of the propyl chains have been eliminated from the molecule. One of these entities is known as isovaleramide. It's structure and activity are described in U.S. Pat. Nos. 5,763,494 and 5,506,268, the contents of both which are hereby incorporated by reference.

Other analogs have been described in U.S. patent application Ser. No. 09/258,882, filed Mar. 1, 1999, the contents of which are hereby incorporated by reference. These structures may be represented by the following formula:

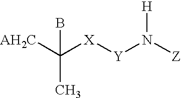

Where:

A═H, CH₃ or OH,

B═H, OH, or CH₃

X═CH₂, CHCH₃, C(CH₃)₂, —O—, CH(OH), or —CH₂O—,

Y═—CO—, or —SO₂—, and

Z═H, CH₂CO₂H, or CH₂CONH₂

Isovaleramide may be represented by the structure above in which Z is H, Y is CO, X is CH₂ and both B and A are H.

Other valproic analogs have been described in U.S. Pat. No. 5,585,358, the contents of which are hereby incorporated by reference. These valproic analogs may be represented by the following formula:

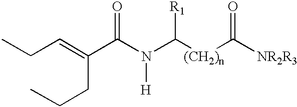

wherein R₁, R₂, and R₃ are independently the same or different and are hydrogen, C₁-C₆ alkyl, aralkyl, or aryl, and n is an integer which is greater than or equal to 0 and less than or equal to 3.

Further valproic analogs have been prepared in which either unsaturation or a cycloalkyl moiety has been incorporated into one of the proply moieties of the valproic acid structure. These compounds are described in U.S. Pat. No. 5,786,380, the contents of which are hereby incorporated by reference. The more preferred compounds of this patent may be represented by the structures below:

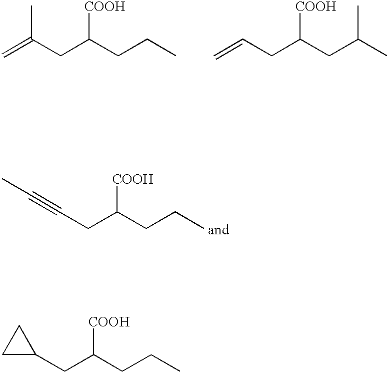

As used in this application, any reference to “valproate compounds” should be construed as including valproic acid, the sodium salt of valproic acid, divalproex sodium, any of the various salts of valproic acid described above, any of the prodrugs of valproic acid described above, and any of the analogs of valproic acids described above. Divalproex sodium is the most preferred valproate compound of the present invention.

As noted above, one aspect of the invention is directed to an improved process for granulating valproate compounds. The resulting product from that process is a granulate. As used in this application, the term “granulate” refers to valproate compounds that have been melted sufficiently to produce granules that are suitable for compressing into tablets. The terms “granulate” and “agglomerate” should be considered as synonyms with this application.

A further aspect of this invention is an improved process for manufacturing solid dosage forms of valproate compounds. Typically, these dosage forms will be tablets. However, the valproic granulates produced by this invention may be incorporated into capsules, lozenges, chewable tablets, sprinkles, particles, etc. As used in this application, the term “solid dosage form” should be construed as covering any non-liquid pharmaceutical composition containing the valproic granulate of this invention, in a form suitable for oral consumption by a human being.

### I. Overview of Process

A generalized overview of the process of this invention is schematically represented below in Scheme I. As would be apparent to one skilled in the art, this process can be modified in a number of ways depending upon the specific granulating equipment available. A more preferred version of this process, using specific types of granulating devices, is described on page 18 of this application.

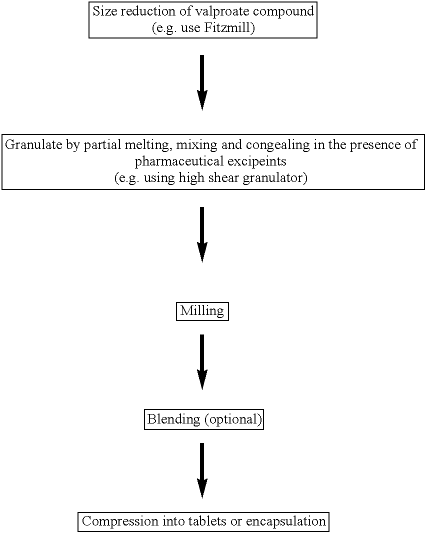

Step A) Sizing

The starting material in the process is the drug substance (i.e. the valproate compounds). This drug substance will be in the form of a bulk powder. Typically, the initial step in the process is to subject the powdered valproate compound to a milling or screening step. This is done in order to reduce the particle size of the valproate compound. Many techniques are known in the art for accomplishing such a result. They are described in detail in Remington's Pharmaceutical Sciences, Fifteenth Edition, pages 1584-1585, (1975). Small batches can be forced by hand through a manual screen. Large quantifies can be forced through sieving devices such as a Stokes oscillator, a Colton rotary granulator, a Stokes mill, a Fitzpatrick mill, etc. The valproate compounds should be milled to a particle size of less than 4760 μm and more preferably less than 210 μm.

Step B) Granulation

The next step of the process is the granulation step. In this step, the valproate compound is introduced into a container and is heated to, or approaching, the melting point for that specific compound. It is not necessary to melt all of the valproate compound in order to achieve the benefits described above. Typically, at least 10 w/w % of the valproate compound will be melted (based upon the total weight of the valproate compound contained within the container). More preferably about 25 w/w % will be melted and most preferably about 40 w/w % will be melted. As used herein, a valproate compound is considered to be melted or in a molten state when it is a liquid.

The temperature required to melt the valproate compounds will vary depending upon the specific compound. As a general guideline, a temperature of from about 70° C. to about 150° C. will be required. More preferably, a temperature in the range of 90° C. to 130° C. will be used. For example with divalproex sodium, a temperature in the range of from about 100° C. to about 140° C. will be used, and more preferably one in the range of from about 110° C. to about 130° C.

The time required to melt the valproate compound will also vary depending upon the specific valproate compound, the quantity involved, the temperature used, the rate of stirring, the specific container or device in which the granulation is carried out in, etc. Manipulating these multiple variables in order to achieve a successful granulation can be readily accomplished by one skilled in the art. As a general guideline, the valproate compound will be heated for a period of time ranging from 5 minutes to 120 minutes, and more preferably 60 minutes to 90 minutes. As a general guideline, it typically takes about 30-90 minutes to melt a kilogram of valproate compound, once the melting point of that specific compound is reached.

The container, or device, the granulation is carried out in is not critical. Any container capable of both heating and agitating the valproic admixture may be utilized. A variety of such devices exist and are well known to those skilled in the art. Examples of suitable granulation devices include jacketed high shear mixers,jacketed mixing tanks, etc. The rate or speed at which the valproic admixture is agitated, mixed or stirred can vary widely. As a general guideline, the rate of agitation is decreased as the volume of granulate increases. A speed of about 430-650 rpm is suitable for a volume of 10 liters, a speed of about 70-300 rpm is suitable for a volume of 75 liters, and a speed of about20-185 rpm is suitable for a volume of 300 liters.

As noted above, it is necessary to melt a substantial quantity of the valproate compound in order to achieve a suitable granulate. The melted valproate compound serves as it own binding agent. It serves to create the cohesiveness between the valproic particles and the excipeints that is required to produce a granulate capable of being compressed into a tablet. It is this aspect of the invention that differentiates it from the prior art hot melt granulation techniques. The prior art teaches that a separate binder is required. Further this binder should have a melting point substantially below the melting point of the drug being granulated. Melting a drug often leads to its degradation and decrease of biological activity. Melting the valproate compounds has been found not to have any adverse impact upon their biological activity.

After a sufficient quantity of the valproate compound has been melted, the heating may be terminated. The admixture should then be mixed or stirred until it has cooled to room temperature and congealed. This is typically accomplished by leaving the admixture in the granulator and continuing the stirring while cooling. The time required to accomplish this result will vary depending upon the quantity of the valproate compound, the heat conduction capacity of the granulator, the specific valproate compound, etc. Typically, it will take from 5 minutes to 40 minutes before the mixture congeals.

As is well known to those skilled in the art, pharmaceutical excipeints are routinely incorporated into solid dosage forms. This is done to ease the manufacturing process as well as to improve the performance of the dosage form. These excipeints will be incorporated into the solid dosage forms of this invention and are typically introduced prior to the granulation step. Common excipeints include diluents or bulking agents, lubricants, disintegrants, etc.

Diluents are added in order to increase the mass of an individual dose to a size suitable for tablet compression. Suitable diluents include powdered sugar, calcium phosphate, calcium sulfate, microcrystalline cellulose, lactose, mannitol, kaolin, sodium chloride, dry starch, sorbitol, etc. If a chewable tablet is desired, then mannitol, lactose, sorbitol, or inositol will be utilized.

Lubricants are incorporated into a formulation for a variety of reasons. They reduce friction between the granulation and die wall during compression and ejection. This prevents the granulate from sticking to the tablet punches, facilitates its ejection from the tablet punches, etc. Examples of suitable lubricants include talc, stearic acid, vegetable oil, calcium stearate, zinc stearate, magnesium stearate, etc.

Glidant's are also typically incorporated into the formulation. A glidant improves the flow characteristics of the granulation. Examples of suitable glidant's include talc, silicon dioxide, and cornstarch.

Disintegrants are also typically incorporated into the formulations. They facilitate the break-up or disintegration of the tablet after it has been ingested by the patient. Examples of suitable disintegrating agents include starch and starch compounds such as, sodium starch glycolate, celluloses and cellulosic compounds, cross-linked polyvinylpyrrolidone, agar, bentonite, guar gum, microcrystalline cellulose, pregelatinized corn starch, croscarmalose, etc.

Other excipeints that may be incorporated into the formulation include meltable excipeints such as waxes, glycerides, polyethylene glycol, carbomer, or antioxidants, preservatives etc.

The excipeints may be incorporated into the dosage forms using any of the techniques known in the art. Typically, they will be dry blended with the valproate compound prior to the granulation. This may be done using techniques known in the art such as dry blending in a high shear mixer, v-blender, bin blender, etc.

The quantity of excipeints can vary widely; but as mentioned above, one of the advantages of this invention is that it allows the production of solid dosage forms containing substantially greater quantities of the valproate compound, than is possible using the prior art methods. It is possible to produce tablets which contain at least 75% by weight of the valproate compound and up to about 90% by weight of the valproate compound, based upon the total weight of the tablet. To gain such benefits, the excipeints will compose at most the remaining 10-25 w/w % of the formulation. More preferably, the excipeints will be present in the dosage form in a quantity ranging from about 20% w/w to about 30% w/w. The specific amount of each of the various types of excipeints can vary widely within these guidelines. Table I below provides guidelines for the quantities of excipeints that may be utilized for the formulations.

TABLE 1
Quantity Guidelines (w/w % based upon total formulation wt)

| Substance | Typical | Preferred | Most Preferred |
|---|---|---|---|
| Valproate compounds | 50-90% | 65-85% | 75-80% |
| Glidant | 1-6% | 2-5% | 3-4% |
| Diluent | 10-50% | 15-35% s | 20-25% |
| Disintegrant | 10-20% | 8-15% | 5-10% |
| Lubricants | 0.5-3.0% | 0.5-2.0% | 0.5-1.5% |

As will be described in greater detail below, the solid dosage forms of this invention may be coated. The quantities of valproate compound and excipeints above refer to the amounts of these ingredients in any uncoated cores, and not to the amounts in a dosage form which has been coated. The coating should be considered additive to the cores and not included in any drug loading calculation. For example, a 105 mg tablet containing 75 mg of divalproex sodium, 25 mg of excipeints, and 5 mg of coating; should be considered to have 75% of divalproex sodium and 25% of excipeint (drug loading factor of 75%). This 105 mg tablet before or after it has been coated should be considered a solid dosage form for the purposes of this invention including the claims attached hereto.

Step C) Milling

Once the granulation is completed, the congealed granulate is removed from the container and milled to produce particles suitable for compression into tablets or encapsulation. This can be accomplished using the same techniques as described in Step A. A particle size in the range of 44 μm to 1200 μm should be obtained.

Step D) Optional Blending

After the milling has been accomplished, additional pharmaceutical excipeints may be introduced into the granulate for incorporation into the finished dosage form as described above. Such a step is not required and can vary depending upon the properties desired for the final dosage form. The quantity of any excipeint added should be within the guidelines described above. Typically though, a glidant is added at this step of the process. Other excipeints that may be incorporated at this step include lubricants or disintegrants.

Any additional excipeints can be introduced into the granulate using techniques well known to those skilled in the art. Typically they will be introduced via dry blending. Suitable dry blending devices include v-blenders or bin blenders.

Step E) Preparation of the Dosage Form

The final step of the process is the production of the solid dosage form. Typically, the granulate will be compressed directly into tablets. However, the granulate may also be filled into capsules, saches, lozenges, etc.

Methods for producing these solid dosage forms are well known to those skilled in the art. Methods for producing tablets are described in Remington's, supra at pages 1576-1598, the disclosure of which is hereby incorporated by reference. As a general guideline, a predetermined quantity of the granulate is placed in a steel cavity (lower punch). An upper punch is lowered into the steel cavity exerting pressure on the granulate thereby forming the tablet. The pressure required to form the tablet can vary widely depending upon the punch utilized, the desired tablet hardness, dissolution rate, etc. However, as a general guideline a pressure in the range of about 7-35 kilo newtons (KN) will be utilized. Examples of suitable tablet punches include oval, concave, and round.

If desired, the tablets can be coated as is known in the art. Tablets are coated to improve their appearance, protect them from atmospheric degradation, control the site of drug release (i.e. enteric coatings), delay or prolong their release patterns, etc. Methods for coating tablets are well know in the art and have been described in detail by Remington's at pages 1608-1617, supra, the disclosure of which is hereby incorporated by reference. The quantity of such coating can vary widely depending upon the reason for the coating. One skilled in the art can readily determine how much coating should be applied to the tablets in order to produce a desired result.

The granulation techniques described above are suitable for immediate release or delayed release tablets. As is know to those skilled in the art, polymers are incorporated into formulations to delay or extend their release patterns. One skilled in the art could adjust the granulation process described above to produce such delayed or extend release dosage forms.

In addition to tablets, the granulate can also be incorporated into capsules. Method for producing capsules are also well known in the art. Such methods are described in detail by Remington's, supra, at pages 1598-1605, the disclosure of which is hereby incorporated by reference. As a general principle, the granulate is prepared as described above, and then filled into gelatin capsules as is known in the art. A variety of automatated capsule filling machines are available to accomplish this result. Examples of suitable devices include Zanasi, MG-2, and Hoefliger & Karg models.

### II. NEW DOSAGE FORMS

As has been previously described earlier in this application, one advantage of this invention is that it allows the production of solid dosage forms containing a greater concentration of the valproate compound in a given volume. This concept is referred to as drug loading by those skilled in the art. Drug loading refers to the quantity of drug contained within the dosage form expressed as a percentage of the total weight of the dosage form (i.e. w/w %). The process of this invention allows the production of dosage forms containing at least 75 w/w % of the valproate compound and up to 90%. More preferably the valproate compound will be present in a quantity ranging from about 75 w/w % to about 80 w/w %. The prior art wet granulation process only allow for the production of valproic formulations having a maximum drug loading factor of 63 w/w %.

Increasing drug loading allows the production of solid dosage forms that are substantially smaller than those using the processes of the prior art. For example, divalproex sodium is commercially available in the United States as tablets in dosages of 125 mg, 250 mg, and 500 mg. Depending upon the disease being treated, the total daily dose can reach 4 grams, consumed in 2 to 8 separate daily doses. This requires the patient to take multiple tablets to achieve only one dose. Patients often have to consume from 2 to 6 tablets per day. It is often difficult for patients to remember to consume such a large number of tablets, especially when they must consume tablets of different strength to obtain a single dose (1 dose contains a 500 mg tablet and a 250 mg tablet). This leads to doses being missed and ultimately sub-therapeutic blood levels of the drug. Thus it would be extremely desirable to produce a 750 mg divalproex sodium tablet, thereby decreasing the number tablets a patient must consume in a given day. Despite this apparent need, current granulation processes do not allow such a result. A 750 mg divalproex sodium tablet produced via the prior art processes is too large to be comfortably swallowed by most consumers. The tablet is so large that it can cause a gagging reflex in these patients, which is unacceptable in the marketplace.

The process of this invention solves this problem. It allows the production of a 750 mg divalproex sodium tablet that can be swallowed by most consumers. In order to further exemplify the invention, Table II below gives a size comparison of the tablets produced via the prior art and that produced via the inventive process.

TABLE II
Divalproex Sodium

| Wet Granulation | | | Hot Melt Granulation | | |
|---|---|---|---|---|---|
| Dose | Wt. | Diameter | Dose | Wt. | Diameter |
| (mg) | (mg) | (mm) | (mg) | (mg) | (mm) |
| 500 | 858 | 20 × 10 × 8 | 500 | 600-750 | 17 × 8 × 7 |
| | | | | preferably about | |
| | | | | 650-725 | |
| 750 | n/a | n/a | 750 | 900-1100 | 19 × 9 × 8 |
| | | | | preferably about | |
| | | | | 940-1050 | |

A further advantage of this invention is that it allows the production of solid dosage forms of valproate compounds in which the content of organic solvent is below that which can be detected by the human sense of smell or taste. The granulations of the prior art utilize organic solvents. Residual amounts of these organic solvents are retained in the granulate despite the attempts to remove such solvents during the drying step. The dosage forms produced from these granulates contain sufficient quantities of the organic solvent to produce an odor that is considered unpleasant by many consumers. These wet granulations of the prior art are described in more detail in Process For Producing Solid Doseage Forms of Valproate Compounds, Qiu et al, filed concurrently herewith, which is hereby incorporated by reference.

The human olfactory gland is surprisingly sensitive to the presence of organic solvents. A typical human can detect the presence of organic solvents in relatively minute amounts. For example, humans can detect ethanol by smell at a level of 100 parts per million in an aqueous solution, or acetone at a concentration of 500 part per million in an aqueous solution. The values at which humans can detect common organic solvents, by both taste and smell, has been studied in detail. These values have been compiled on the internet at www.leffingwell.com/odorthre.htm, the contents of which are hereby incorporated by reference.

The solid dosage forms produced via this invention will have an organic solvent content of less than 0.2 w/w %, more preferably less than 0.1 w/w %, and most preferably less than the limit of detection as measured by static headspace gas chromatography. As used in this application, any reference to an organic solvent quantity refers to a level determined by static headspace gas chromatography using the method of the European Pharmacopoeia Supplement (2.2.28) page 12 (2001) as exemplified in Example 7.

### III. PREFERRED PROCESS

In a more preferred embodiment, a specific granulating technique is employed that is referred to as spray congealing. Spray congealing is described in detail by Ghebre-Sellaissie et. al. in Multiparticulate Oral Drug Delivery, Drugs and the Pharmaceutical Sciences, Vol. 65, pages 17-34, which is hereby incorporated by reference. In this process, a drug is dispersed in hot melts of binders such as gums, waxes, etc., which produces a flowable liquid. This liquid admixture is then dispersed or sprayed into an air chamber where the temperature is maintained below the melting point of the binders, thereby congealing the mass which produces spherical congealed pellets.

In the most preferred embodiment of the invention, a specific spray congealing device is utilized. This granulation device is referred to as a spinning disk and is described in detail in U.S. Pat. No. 4,675,140, the contents of which are hereby incorporated by reference. A spinning disk differs from spray congealing in that the molten admixture is contacted with a rotating surface rather than being pumped into an air chamber. The centrifugal force of the rotating surface disperses the admixture into relatively small droplets which congeal as described above.

The use of spray congealing requires minor modifications to the process described above in Scheme I. Scheme II below illustrates this new process:

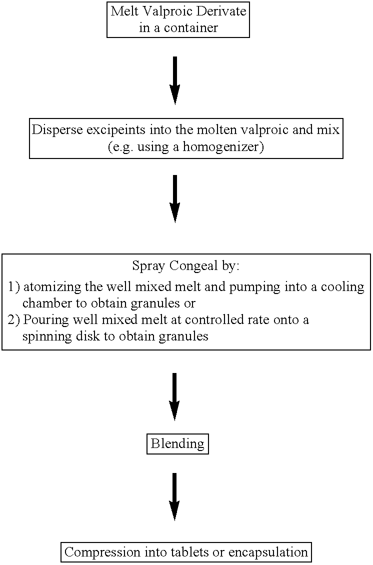

Step A) Melting

As with the process outlined in Scheme I, it is necessary to melt the valproate compound in order to carry out the granulation. The quantity of the valproate compound that must be melted varies however. With this preferred process, substantially all of the valproate compound must be melted. As used herein, “substantially all” refers to at least 80 w/w % of the valproate compound (based on the total weight of the valproate compound) and more preferably from 95 w/w % to 100 w/w %. Melting such large quantities of the valproate compound negates the need for a milling step.

The melting can be carried out in the same manner as described above for Scheme I. In the interest of brevity, this disclosure will not be repeated here. The valproate compound will be heating to its melting point and the heating will continue until substantially all of the valproate compound has been rendered to a molten state.

Step B) Incorporation of Excipeints

The next step in the process is the incorporation of the pharmaceutical excipeints into the molten valproate compound. The same pharmaceutical excipeints will be utilized as described above in Scheme I. Further, the excipeints will be utilized in the same quantities as described above. They will be incorporated into the molten valproate compound in the same manner as described above, except that the heating will not be terminated once the admixture is completed. The heating will be continued throughout the spray congealing process as described below.

Step C) Granulation via Spray Congealing

Any of the spray congealing devices known in the art can be used in this step. All of the devices work by a common principle of transforming a suspension into discrete particles by placing the suspension into motion in an environment having a reduced temperature relative to the suspension. The motion and temperature differential produce discrete granules which may then be further processed in solid dosage forms. This can be accomplished by atomizing the suspension and spraying it into a chamber or by contacting the suspension with a rotating surface as described in greater detail below.

Spray congealing using a spinning disk is described in detail below. However, the invention should not be construed as being limited to that specific device. Any spray congealing device known in the art may be used. One skilled in the art can take the teachings below relative to a spinning disk and use their guidance to produce granules with an atomization chamber or any other spray congealing device. As with a spinning disk, temperature and viscosity must be controlled in order to produce optimum granulates. The teachings below would allow one skilled in the art to accomplish this result.

Spinning disks and their methods of operation are well known in the art. Spinning disks and their use in granulation processes are described in U.S. Pat. Nos. 4,675,140; 5,100,592; and 5,143,662, the contents of all, which are hereby incorporated by reference. Spinning disks operate via the following principle. A suspension of one or more materials is placed onto a rotating disc. The suspension is centrifugally dispersed by the rotating disc into relatively small droplets of the suspended material. As the droplets are dispersed by the rotating disc, they are solidified by exposure to air. The solidified particles are then collected, separated based upon size and transformed into pharmaceutical dosage forms as is known in the art.

As their name implies, spinning disks are substantially circular rotating disks. They may be flat, convex, concave, or even bell-shaped, and can contain protruding vanes on a surface thereof Any of the spinning disks routinely used in the pharmaceutical industry may be used in this invention. Preferably the spinning disk will be flat.

In the process of this invention, the molten admixture of the valproate compound and the excipients are fed onto the rotating disc. The feed rate of the suspension, the viscosity of the suspension, the temperature of the disc and the rotational speed of the disc should be maintained in order to produce a granulate having a diameter in the range of about 20-400 mesh and more preferably about 40-200 mesh.

The size of the spinning disk itself and the rpm, i.e. rate of rotation, can be interrelated to provide the optimum centrifugal acceleration for the formation of the granulate. Variations of this centrifugal acceleration will affect the ultimate size of the particles that are formed. The revolutions of the spinning disk are controlled within a range of about 500-6000 rpm, preferably within a range of about 1000-4000 rpm, and more preferably within a range of about 2000-3000 rpm. Furthermore, the disk itself is preferably between about 20-30 cm in diameter, but can vary widely as is known in the art. The temperature of the spinning disk itself will be managed during the granulation process. The temperature of the spinning disk should be maintained in the range of about 80-130° C. and more preferably about 90-110° C.

The molten valproate compound will typically be maintained within a viscosity range of about 500-20,000 centipoise. The valproate compound will be fed onto the disc at a rate that varies with temperature and viscosity, but that ensures that the molten admixture will be supplied in the form of a thin film onto the surface of the spinning disk, whereby the centrifugal acceleration breaks the thin film into particles of the granulate. Surface tension will cause the resulting particles of the valproate compounds to ultimately harden into particles which are spheroidal or in the shape of beads, as these particles are radically discharged from the disk, i.e. fall off the edge of the rotary spinning disk and are cooled.

The particles of valproate compound are allowed to fall off the edge of the spinning disk and are cooled, e.g., in air, before striking a collecting unit without being deformed. The spinning disk is preferably placed at a predetermined height to allow the spheroidally shaped particles to sufficiently air cool before striking the collecting unit, e.g. collecting trays, so that the particles or beads are not deformed upon striking the collecting unit and will not stick together when collected. The particles are allowed to cool to room temperature. The exact height required to accomplish this result can vary widely as is known to those skilled in the art, but can readily be accomplished to obtain granules suitable for further processing into pharmaceutical dosage forms. The collecting unit may be provided, e.g., as disclosed in U.S. Pat. Nos. 4,256,677 and 3,743,464 both of which are hereby incorporated by reference.

As noted above, granulate formed by the present process lies within a relatively narrow range of particle size. The average particle size of the particles of granulate formed herein is within the range of about 30-200 mesh, preferably within the range of about 40-100 mesh, and more preferably within the range of about 40-80 mesh. This granulate is then subjected to an optional blending step and are transformed into pharmaceutical dosage forms as described below.

Step D) Optional Blending

The granulate produced above may be dry blended with additional excipeints beyond those added above in Step B. Any such excipeints will be the same as those described in Step D of Scheme I and will be incorporated into the granulate in the same manner.

Step E) Preparation of Dosage Form

Once the granulate has been formed as described in Step C and optionally mixed with any further excipeints, it may be manufactured into dosage forms such as tablets, capsules, etc. This may be accomplished in exactly the same manner as described above in Scheme I. These dosage forms also have the advantage of an enhanced drug loading factor as described above.

The following examples are being presented in order to further illustrate the invention. While they exemplify the production of dosage forms containing divalproex sodium, one skilled in the art could apply the teachings of these examples to other valproate compounds. These examples should not be construed in any manner as limiting the scope of the invention, or the claims attached hereto.

### EXAMPLES

### Example 1

Divalproex sodium flakes are weighed, transferred into and melted in a jacketed vessel utilizing a pressurized water bath at a temperature of 130° C. Excipients (Avicel PH101, Sodium Starch Glycolate) are charged in the vessel and mixed well using a motorized homogenizer. The spinning disk is brought to a temperature of 130° C. and a speed of 3000 rpm. The molten liquid is poured or pumped onto the rapidly spinning disk to produce droplets which congeal into small particles. These particles are collected, sifted through a 18 mesh screen and added to a V-blender with Syloid and blended until uniform. The resulted blend is compressed on a rotary tablet press into 720 mg tablets that contain 500 mg valproic acid equivalent or 1080 mg tablets that contain 750 mg valproic acid equivalent. The tablet compositions are given in Table 1.

TABLE 1

| Item | Ingredients | Code | mg/tab | % Load |
|---|---|---|---|---|
| 1 | Divalproex Sodium | 65476 | 537.84 | 74.7 |
| 2 | Sodium Starch Glycolate | 66851 | 72.00 | 10.0 |
| 3 | Microcrystalline Cellulose, | 19615 | 88.56 | 12.3 |
| | Avicel PH101 | | | |
| 4 | Silicon Dioxide (Syloid 244 FP) | 63337 | 21.60 | 3.0 |

### Example 2

Divalproex sodium flakes are weighed, transferred into and melted in a jacketed vessel utilizing a pressurized water bath at a temperature of 130 C. Excipient (Syloid 244) is charged in the vessel and mixed well using a motorized homogenizer. The spinning disk is brought to a temperature of 130-150° C. and a speed of 3000-5500 rpm. The molten liquid is poured or pumped onto the rapidly spinning disk to produce droplets which congeal into small particles. These particles are collected, sifted through a 18 mesh screen and added to a V-blender with Syloid and blended until uniform. Prosolv 90 (silicified mycrocrystalline cellulose) will then be added to the same V-blender containing Syloid and Divalproex sodium and blend until uniform. The resulted blend is compressed on a rotary tablet press into 672-720 mg tablets that contain 500 mg valproic acid equivalent or 1010-1080 mg tablets that contain 750 mg valproic acid equivalent. The tablet compositions are given in Table 2, 3 and 4.

TABLE 2

| Item | Ingredients | Code | mg/tab | % Load |
|---|---|---|---|---|
| 1 | Divalproex Sodium | 65476 | 538.2 | 75 |
| 2 | Silicon Dioxide (Syloid 244 FP) | 63337 | 28.7 | 4 |
| 3 | Silicon Dioxide (Syloid 244 FP) | 63337 | 21.53 | 3 |
| 4 | Prosolv 90 (silicified | N/A | 129.17 | 18 |
| | mycrocrystalline cellulose) | | | |
| total | | | 717.60 | 100 |
| Speed | 5500 rpm | | | |

TABLE 3

| | Ingredients | Code | mg/tab | % Load |
|---|---|---|---|---|
| 1 | Divalproex Sodium | 65476 | 538.2 | 75 |
| 2 | Silicon Dioxide (Syloid 244 FP) | 63337 | 86.11 | 12 |
| 3 | Silicon Dioxide (Syloid 244 FP) | 63337 | 21.53 | 3 |
| 4 | Prosolv 90 | N/A | 71.76 | 10 |
| total | | | 717.60 | 100 |
| speed | 3500 rpm | | | |

TABLE 4

| Item | Ingredients | Code | mg/tab | % Load |
|---|---|---|---|---|
| 1 | Divalproex Sodium | 65476 | 538.2 | 80 |
| 2 | Silicon Dioxide (Syloid 244 FP) | 63337 | 40.37 | 6 |
| 3 | Silicon Dioxide (Syloid 244 FP) | 63337 | 20.18 | 3 |
| 4 | Prosolv 90 | N/A | 74 | 11 |
| total | | | 672.75 | 100 |
| speed | 5500 rpm | | | |

### Example 3

Divalproex sodium flakes are weighed, transferred into and melted in a jacketed vessel utilizing a pressurized water bath at a temperature of 130° C. Excipient (Sodium Starch Glycolate) is charged in the vessel and mixed well using a motorized homogenizer. The spinning disk is brought to a temperature of 130-150° C. and a speed of 3000-5500 rpm. The molten liquid is poured or pumped onto the rapidly spinning disk to produce droplets which congeal into small particles. These particles are collected, sifted through a 18 mesh screen and added to a V-blender with Syloid and blended until uniform. Prosolv 90 will then be added to the same V-blender containing Syloid and Divalproex sodium and blend until uniform. The resulted blend is compressed on a rotary tablet press into 720 mg tablets that contain 500 mg valproic acid equivalent or 1080 mg tablets that contain 750 mg valproic acid equivalent. The tablet compositions are given in Table 5 and 6.

TABLE 5

| Item | Ingredients | Code | mg/tab | % Load |
|---|---|---|---|---|
| 1 | Divalproex Sodium | 65476 | 538.2 | 75 |
| 2 | Sodium Starch Glycolate | 66851 | 28.7 | 4 |
| 3 | Silicon Dioxide (Syloid 244 FP) | 63337 | 21.53 | 3 |
| 4 | Prosolv 90 | N/A | 129.17 | 18 |
| total | | | 717.60 | 100 |
| speed | 3500 rpm | | | |

TABLE 6

| Item | Ingredients | Code | mg/tab | % Load |
|---|---|---|---|---|
| 1 | Divalproex Sodium | 65476 | 538.2 | 75 |
| 2 | Sodium Starch Glycolate | 66851 | 86.11 | 12 |
| 3 | Silicon Dioxide (Syloid 244 FP) | 63337 | 21.53 | 3 |
| 4 | Prosolv 90 | N/A | 71.76 | 10 |
| total | | | 717.60 | 100 |
| speed | 5500 rpm | | | |

### Example 4

Divalproex sodium flakes are weighed, transferred into and melted in a jacketed vessel utilizing a pressurized water bath at a temperature of 130° C. Excipient (Talc) is charged in the vessel and mixed well using a motorized homogenizer. The spinning disk is brought to a temperature of 130-150° C. and a speed of 5500 rpm. The molten liquid is poured or pumped onto the rapidly spinning disk to produce droplets which congeal into small particles. These particles are collected, sifted through a 18 mesh screen and added to a V-blender with Syloid and blended until uniform. Prosolv 90 will then be added to the same V-blender containing Syloid and Divalproex sodium and blend until uniform. The resulted blend is compressed on a rotary tablet press into 670 mg tablets that contain 500 mg valproic acid equivalent or 1010 mg tablets that contain 750 mg valproic acid equivalent. The tablet compositions are given in Table 7.

TABLE 7

| Item | Ingredients | Code | mg/tab | % Load |
|---|---|---|---|---|
| 1 | Divalproex Sodium | 65476 | 538.2 | 80 |
| 2 | Talc | 71200 | 43.06 | 6 |
| 3 | Silicon Dioxide (Syloid 244 FP) | 63337 | 21.53 | 3 |
| 4 | Prosolv 90 | N/A | 78.94 | 11 |
| total | | | 672.75 | 100 |
| speed | 5500 rpm | | | |

### Example 5

Divalproex sodium flakes are weighed, transferred into and melted in a jacketed vessel utilizing a pressurized water bath at a temperature of 130° C. Excipient (Fujicalin) dicalcium phophate is charged in the vessel and mixed well using a motorized homogenizer. The spinning disk is brought to a temperature of 130-150° C. and a speed of 5500 rpm. The molten liquid is poured or pumped onto the rapidly spinning disk to produce droplets which congeal into small particles. These particles are collected, sifted through a 18 mesh screen and added to a V-blender with Syloid and blended until uniform. Prosolv 90 will then be added to the same V-blender containing Syloid and Divalproex sodium and blend until uniform. The resulted blend is compressed on a rotary tablet press into 670 mg tablets that contain 500 mg valproic acid equivalent or 1010 mg tablets that contain 750 mg valproic acid equivalent. The tablet compositions are given in Table 8.

TABLE 8

| Item | Ingredients | Code | mg/tab | % Load |
|---|---|---|---|---|
| 1 | Divalproex Sodium | 65476 | 538.2 | 80 |
| 2 | Fujicalin dicalcium phophate | N/A | 43.06 | 6 |
| 3 | Silicon Dioxide (Syloid 244 FP) | 63337 | 21.53 | 3 |
| 4 | Prosolv 90 | N/A | 78.94 | 11 |
| total | | | 672.75 | 100 |
| speed | 5500 rpm | | | |

### Example 6

This example illustrates the results of in vitro dissolution testing on a dosage form prepared using the methods described above in Example 1-5.

In vitro dissolution rate of the tablets were compared with that of the reference, depakote, the uncoated marketed tablet, which contains the same amount of the active ingredient. USP apparatus II was used for testing. The test condition was: paddle speed=50 rpm; dissolution medium=900 ml 0.5M phosphate buffer at pH 7.5; temperature=37° C. Dissolution samples were analyzed by a TDX florescence-polarized immunoassay. Results and Discussions:

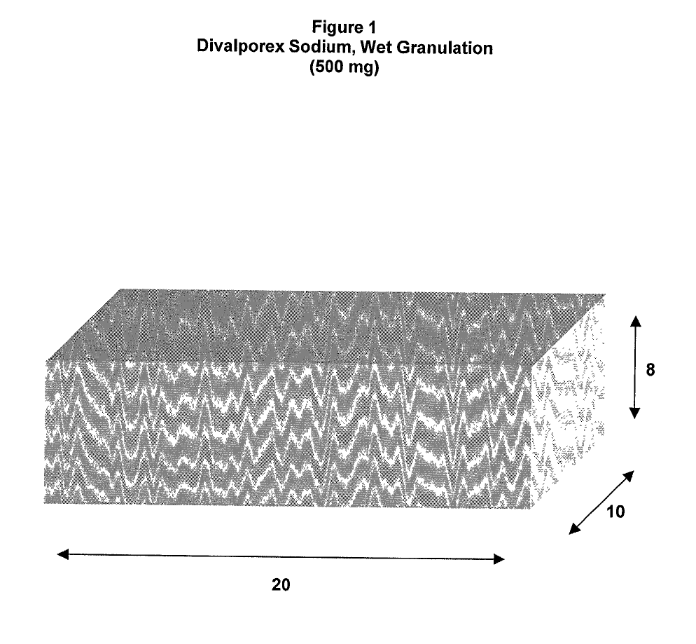

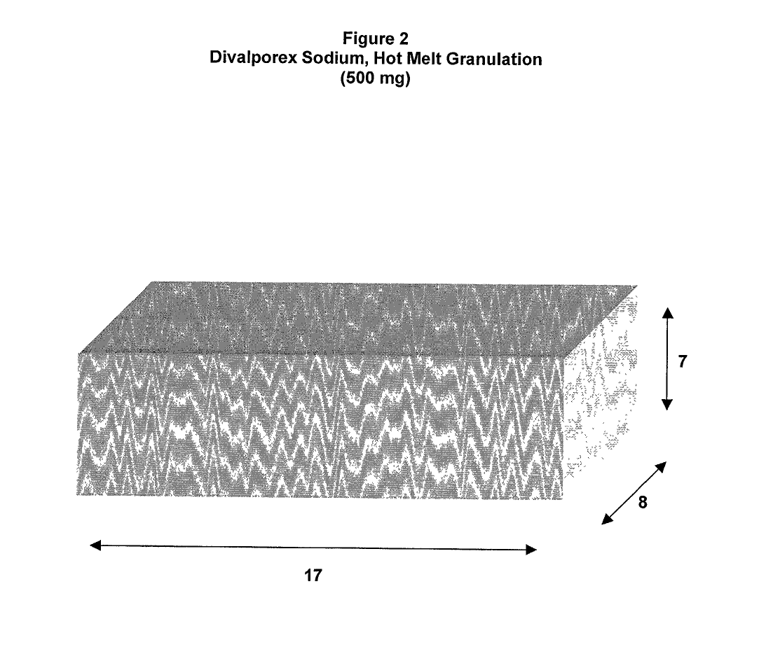

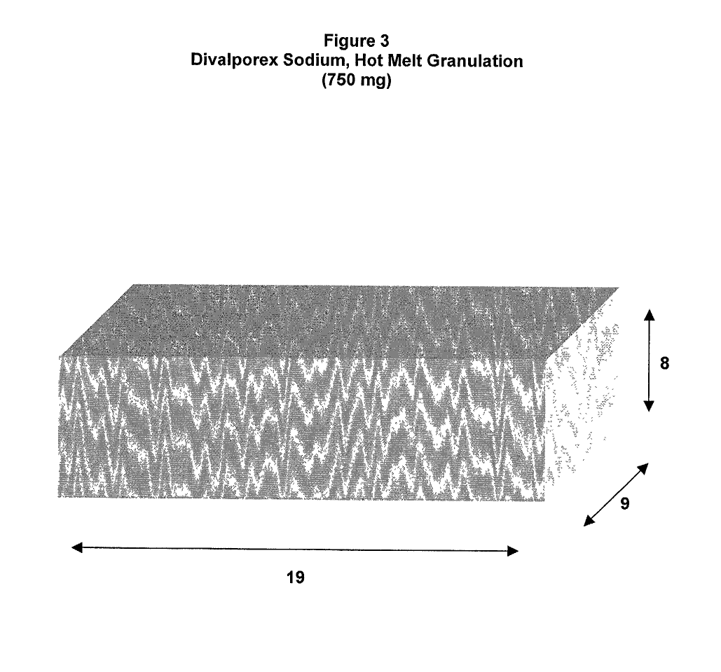

In vitro dissolution profiles of the reference tablet and tablet from the current invention shown in FIG. 1 indicate that dissolution of the current invention is rapid, complete and equivalent to the reference (>90% in 20 minutes). Because Divalproex sodium is a soluble, permeable and stable compound and known to have complete oral absorption, equivalent dissolution in vitro can result in equivalent in vivo absorption.

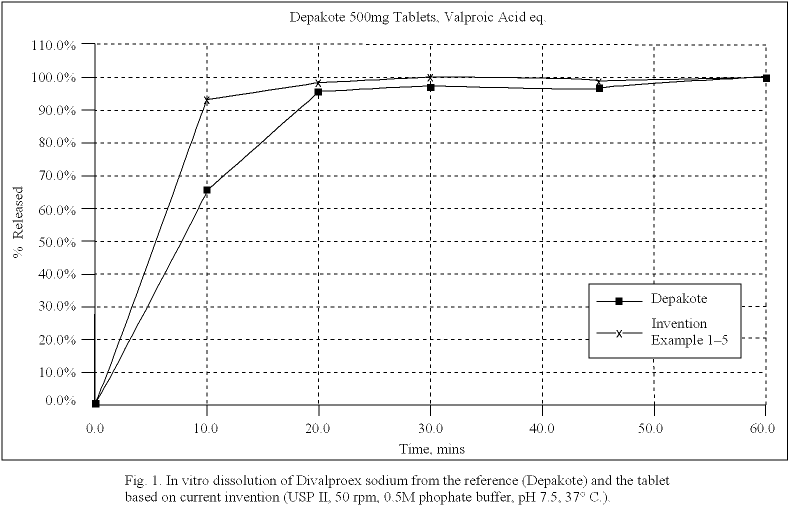

### Example 7

Tablets produced using the methods of Examples 1 and 2 were subjected to static headspace gas chromatography and analysed for the presence of methanol, ethanol and acetone.

Static Headspace Gas Chromatography

As stated in the Ph. Eur. (2.2.28), “Static head-space gas chromatography is a technique particularly suitable for separating and determining volatile compounds present in solid or liquid samples. The method is based on the analysis of the vapour phase in equilibrium with the solid or liquid phase. The sample to be analysed is introduced into a container fitted with a suitable stopper and a valve-system which permits the passage of the carrier gas. The container is placed in a thermostatically controlled chamber at a temperature set according to the substance to be examined. The sample is held at this temperature long enough to allow equilibrium to be established between the solid or liquid phase and the vapour phase. The carrier gas is introduced into the container and, after the prescribed time, a suitable valve is opened so that the gas expands towards the chromatographic column taking the volatilised compounds with it.”

Testing Instructions for the Determination of Ethanol, Acetone, and Methanol in Depakote 500 mg IR Tablets, by Static Headspace Gas Chromatography:

Residual Solvents

Examine by gas chromatography [Ph. Eur. (2.2.28)], using a head space injector and vials which can be crimp-sealed and which are compatible with the injector.

Reference Solution

Dilute 110 mg of ethanol (R) in dimethylformamide (R) and make up to 50.0 mL with the same solvent=solution (1)

Dilute 50 mg of acetone (R) in dimethylformamide (R) and make up to 100.0 mL with the same solvent=solution (2)

Dilute 50 mg of methanol (R) in dimethylformamide (R) and make up to 100.0 mL with the same solvent=solution (3)

In a volumetric flask of 100 mL, introduce 25.0 mL of solution (1), 10.0 mL of solution (2), 10.0 mL of solution (3) and make up to volume with dimethylformamide (R).

In a vial which is compatible with the injection system, introduce 1.0 mL of the reference solution and crimp-seal the vial.

Test Solution

Place 2 tablets, coarsely ground in a 50 mL glass vial with grinded stopper and add 20.0 mL of dimethylformamide (R). Shake 5 minutes on a magnetic agitator and 10 minutes with ultrasonic waves. Allow the solution to place and introduce 1.0 mL of the supernatant in the vial which is compatible with the injection system and crimp-seal the vial.

The reagents (R) are described in the Ph. Eur.

Injection Sequence

1. Inject the reference solution 6 times, in a manner which brackets the test solution. Inject the test solution.

Chromatographic Conditions and Equipment

Hewlett Packard 7694 Headspace GC

| Zone temperatures | |
|---|---|
| Oven-90° C. | Shaker setting-Off |
| Loop-110° C. | |
| Transfer line-120° C. | Pressure-20 psi |
| Timed events | |
| GC cycle time-30 minutes | |
| Inject time-0.5 minutes | Injection volume-1 mL |
| Loop equilibration-0.03 minute | |
| Loop fill time-0.15 minutes | |
| Vial Pressurization time-0.12 minutes | Pressure-16.3 psi |
| Vial equilibration time-15 minutes | |

Hewlett Packard 5890 Series II Plus GC

Injection temp-200° C.

Injection type-Split; split flow: 50 mL/min

Oven program 110° C. for 8 minutes, ramp 20° C./min to 240° C. for 6 minutes; 2 minute oven equilibration

Detector temp-270° C.

Helium flow inlet B-16 psi (3.7 mL/min)

Column: Chrompack Poraplot QPlot FS (10 m×0.32 mm, 10 um film)

Results:

The following results were obtained:

| Solvent | Amt | |
|---|---|---|
| Methanol | <0.0351 | mg/tablet |
| Ethanol | <1.964 | mg/tablet |
| Acetone | <0.1249 | mg/tablet |

We claim:

1. A solid dosage form comprising:
a) about 750 mg of divalproex sodium;
b) one or more pharmaceutically acceptable excipients, and;
c) said solid dosage form has a total weight of about 1,100 mg, or less.

2. A solid dosage form comprising:
a) divalproex sodium, having a drug loading factor of at least 75 w/w %.

3. The solid dosage form according to claim 2 having a drug loading factor in the range of 75-90 w/w %.

4. A process for granulating divalproex sodium comprising:
a) heating said divalproex sodium to a sufficient temperature and for a sufficient period of time to allow a substantial quantity of the divalproex sodium to melt, and;
b) cooling said molten admixture of divalproex sodium under agitation for a sufficient period of time to allow to allow the formation of a granulate suitable for direct compression into a solid dosage form suitable for human consumption.

5. The process according to claim 4 in which said divalproex sodium is heated to a temperature ranging from about 70° C. to about 150° C.

6. The process according to claim 4 in which at least 10 w/w % of said divalproex is melted.

7. The process according to claim 4 in which said granulate is optionally milled and is compressed into a solid dosage form.

8. A process for granulating divalproex sodium comprising:
a) heating said divalproex sodium to a sufficient temperature and for a sufficient period of time to melt substantially all of said divalproex sodium;
b) optionally admixing one, or more, pharmaceutical excipeints with said melted divalproex sodium, and;
c) spray congealing said melted divalproex sodium in a manner suitable for producing granules.

10. The process according to claim 9 in which said spray congealing is accomplished by contacting the molten divalproex sodium with a rotating surface at a rate suitable for producing granules.

11. The process according to claim 9 in which said spray congealing is accomplished by atomizing said molten divalproex sodium and spraying it into an environment having a reduced temperature.

12. The process according to claim 9 in which said granulate is optionally milled and is compressed into a solid dosage form.

13. A solid dosage form comprising:
a) a valproate compound, having a drug loading factor of at least 75 w/w %.

14. The solid dosage form according to claim 13 having a drug loading factor in the range of 75-90 w/w %.

15. A process for granulating valproate compounds comprising:
a) heating said compound to a sufficient temperature and for a sufficient period of time to allow a substantial quantity of the compound to melt, and;
b) cooling said molten admixture of said compound under agitation for a sufficient period of time to allow to allow the formation of a granulate suitable for direct compression into a solid dosage form suitable for human consumption.

16. The process according to claim 15 in which said granulate is optionally milled and is compressed into a solid dosage form.

17. A process for granulating a valproate compound comprising:
a) heating said compound to a sufficient temperature and for a sufficient period of time to melt substantially all of said compound;
b) optionally admixing one, or more, pharmaceutical excipeints with said melted compound, and;
c) spray congealing said melted compound in a manner suitable for producing granules.

18. The process according to claim 17 in which said spray congealing is accomplished by contacting the molten compound with a rotating surface at a rate suitable for producing granules.

19. The process according to claim 17 in which said spray congealing is accomplished by atomizing said molten compound and spraying it into an environment having a reduced temperature.

20. The process according to claim 17 in which said granulate is optionally milled and is compressed into a solid dosage form.
